(19) United States
(12) Patent Application Publication
(10) Pub. No.: US 2003/0175759 A1
(43) Pub. Date: Sep. 18, 2003

(54) Gene predicting method and list providing method

(75) Inventors: Takamichi Muramatsu, Hatoyama (JP); Ayako Yamamoto, Kunitachi (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/309,152

(22) Filed: Dec. 4, 2002

(30) Foreign Application Priority Data
Feb. 25, 2002 (JP) P2002-047297

(51) Int. Cl. C12Q 1/68; G06F 19/00; G01N 33/48; G01N 33/50

(52) U.S. Cl. 435/6; 702/20

(57) ABSTRACT

The invention makes it possible to predict a gene contained in a DNA fragment obtained as a result of gene expression analysis effectively in a simple and easy manner. Searching is made in a gene database utilizing the information about the size from a known nucleotide sequence to a specific sequence in a target fragment and the information about the specific sequence to thereby extract the predicted gene. The invention makes it possible to predict and identify a target fragment gene rapidly, whereby the gene analysis efficiency is markedly improved.

### BACKGROUND OF THE INVENTION

1. Field of the Invention

The present invention relates to a method of gene analysis by which a gene expressed in cells or tissue of a living organism can be analyzed efficiently and, particularly, to a method of searching for a useful or novel gene and a method of preparing a list concerning the predicted gene and providing the list, a method of cloning a predicted gene and providing the cloned products, a method of producing a chip by immobilizing a predicted gene on a chip, and a method of immobilizing a predicted gene on a chip and hybridizing the gene with a gene complementary thereto.

2. Description of the Related Art

In the prior art of gene analysis, a number of mutants have been produced and used for analytical purposes for identifying genes or the loci thereof on chromosomes. In particular, in Arabidopsis thaliana (flowering brassica) among plants, and in Drosophila melanogaster (fruit fly) among animals, mutant analysis has been conducted extensively, so that DNA markers, and restriction enzyme recognition sites, among others, have been mapped in detail in the physical maps of the chromosomes thereof. Using the information from such detail gene maps, identification of the loci of novel genes or linkage analysis has been conducted frequently.

On the other hand, in recent years, the performance characteristics of DNA sequencers have been improved and, as a result, nucleotide sequence determination of genes of various living organisms has been carried out in the form of genome projects. Thus, the information concerning genes has been accumulated rapidly. As a result of these genome projects on various living organisms, it has been revealed that a human being has 30 to 40 thousand genes and there are about 18 thousand genes, about half as compared with human beings, in Drosophila melanogaster.

However, in spite of such accumulation of the nucleotide sequence information, many gene functions remain unknown. Therefore, a post-genomic era of investigating the functions of genes has begun. Gene expression analysis, for instance, may be mentioned as one aspect of such function analysis. It is genes expressed in appropriate cells, in an appropriate stage, and to an appropriate extent that decide the development and the function of the relevant tissue. A gene whose expression is specific to a tissue is very important for the formation of that tissue and the maintenance of a specific function of cells of that tissue.

Finding or identifying a gene which is expressed in excess in a diseased tissue or a gene whose expression is suppressed in such a tissue can lead to elucidation of the cause of a disease or the mechanisms of canceration of cells or growth of cancer cells, for instance, making it possible to treat the relevant disease. For finding such disease-associated gene, a gene whose expression is modified by administration of a drug (drug-responsive gene), or a gene whose expression is modified by an environmental change, for instance, and utilizing such a gene in gene therapy or medicinal science, gene expression profiling is urgently required.

Thus, for example, a technique called differential display (DD) was developed in 1992 by Liang, P. and Pardee, A. B. (Science, 257, 967-971) for simultaneously comparing the difference in gene expression between cell populations under different physiological conditions or between different cell groups of various kinds. According to this technique, polymerase chain reaction (PCR) is carried out using cDNA as a template together with radioisotope (RI)-labeled primers, and the levels of expression of the genes are compared by electrophoresis. Since different regions of genes can be amplified by using a large number of primers and changing the primer combination, all genes expressing in the samples can be analyzed exhaustively by this technique. The fluorescent differential display (FDD) technique is an improvement on the above DD method and uses a fluorescent-labeled primer as one of the primers. Further, the amplified fragment length polymorphism (AFLP) method for searching for a function-related gene by direct comparison of genomic information data, the method comprising preparing an EST database and searching for a function-related gene by randomly sequencing a large number of expressed genes, the technique involving rapid amplification of cDNA ends (RACE) for cloning a full-length gene from the information on partial sequences, and other various methods are in use for examining the functions of genes. In any of those methods, a pool containing a large number of gene fragments is obtained in admixture. For finding out a target gene from among this pool, DNA fragments which are different in size are first separated and purified through a plurality of steps, their nucleotide sequences are determined using a sequencer, and the gene of interest is examined as to whether it is a gene already known or has a highly homologous sequence region thereto or not. As an example of such search, a known method is a homology search, such as BLAST, using the GenBank database. Thus, a gene, whether it is a known one or an unknown one, can be identified only after sequencing.

In the conventional methods for gene identification, DNA fragments obtained through the respective methods of analysis mentioned above are separated by agarose electrophoresis or acrylamide electrophoresis, DNA fragment-containing gel portions are excised one by one, and DNA is extracted from each gel portion by any of various methods. For example, there are available the method consisting in electric extraction (Pun, K. K., and Kam, W. (1990), Prep. Biochem., 20, 123-135) and the method comprising dissolving the gel using sodium iodide (Vogelstein, B., and Gillespie, D. (1979), Proc. Natl. Acad. Sci. U.S.A., 76, 615-619). When the gene extracted by such a method is sufficiently pure, the extracted DNA fragment can be directly sequenced. In most cases, however, cloning by insertion into a vector is required. Furthermore, for confirming that the clone obtained is the desired fragment, it is necessary to confirm the mobility by again carrying out electrophoresis. Further, in the DD method, a plurality of fragments show the same mobility in many cases, causing the problem of false positivity; thus, further purification or some other contrivance is required.

### SUMMARY OF THE INVENTION

The present invention provides a technology of gene analysis by which the working efficiency in such gene identification can be improved and a novel gene or a useful gene can be identified efficiently. Further, a preferred method of gene analysis of the present invention is cost-effective owing to simplification of procedures and can treat a large number of genes or gene fragments simultaneously. Another preferred method of the present invention allows for the efficient utilization of the increasingly abundant genetic information resulting from the expansion of genome projects.

The term “useful gene” as used herein includes, within the meaning thereof, disease-related genes, and marker genes and like genes which can be used in gene diagnosis, and thus includes genes useful in gene therapy or medicinal science. The term also includes genes whose expression is modified by an exogenous factor such as a hormone or a drug, and important genes involved in activities of living organisms. The “novel gene” includes, within the meaning thereof, genes not yet registered in any database and, further, genes or gene fragments more or less longer or shorter than genes registered in some database (namely genes more or less longer or shorter than ESTs registered in some database). The term “novel gene” also refers to a gene for which more data has become available than currently contained in a database.

The preferred methods of the present invention include one or more of the following steps:

(1) First, a target fragment containing a target gene is prepared. The target fragment is a gene fragment to be identified as obtained as a result, or in the process, of various analyses. For example, such gene fragment may comprise a gene fragment showing an expected change in expression level as revealed by comparison between a patient-derived and a normal subject-derived sample, or by comparison between samples taken at different times with respect to a change in response to a stimulus, using the DD method. A gene fragment obtained by the AFLP, RACE or like method can also be used. DNA fragments obtained in the process of various gene analyses, such as a DNA fragment which is the insert in a shotgun cloning product, can also be utilized.

The step of target fragment preparation is now briefly described. PCR can be utilized as a method of obtaining a plurality of gene fragments at the same time and in large amounts. By using a primer comprising a known nucleotide sequence and a terminally labeled primer, it is possible to prepare fragments having the known nucleotide sequence at one end and the labeled sequence at the other in large amounts. When they are inserted into a plasmid or the like, they can be amplified using a host such as Escherichia coli.

A target fragment is selected and is treated for cleavage at a specific sequence. Whether the target fragment has such a specific sequence or not can be revealed by cleavage treatment. When the target fragment has a specific sequence, sequence-specific cleavage treatment gives cleavage fragments for each specific sequence, and the size measurement results for the respective cleavage fragments and the sequence information about this specific sequence can be obtained. Since this specific sequence site information and the cleavage fragment size information are specific to the target fragment, candidate genes can be extracted from an existing gene database utilizing these pieces of information. On the other hand, when the target fragment has no such specific sequence, the target fragment is not cleaved. Therefore, if no cleavage fragment is obtained, information is obtained to the effect that the target fragment has no relevant specific sequence.

The cleavage treatment is preferably and judiciously carried out using a restriction enzyme. Any other treatment capable of causing sequence-dependent cleavage, for example ribozyme treatment or chemical treatment, may also be employed. The term “specific sequence” as used herein means that specific nucleotide sequence in a continued nucleotide sequence which is specifically recognized and cleaved by a specific restriction enzyme or a nucleic acid-cleaving enzyme other than a restriction enzyme, an artificially prepared ribozyme or the like.

In cases where one end of the target fragment has a known nucleotide sequence and either end thereof has a label, the cleavage fragments produced by the above sequence-specific cleavage treatment can be recognized with ease. As a result of each sequence-specific cleavage of the target fragment at a plurality of specific sequences, information is obtained about the presence or absence of specific sequences in the target fragment, together with the size information about the distances from the known nucleotide sequence to the respective specific sequences. When the labeled end and the known nucleotide sequence are one and the same end, the distance between the above known nucleotide sequence and the specific sequence can be directly obtained. On the other hand, when the label occurs at the other end opposite to the known nucleotide sequence, the size of the above fragment (distance from the known nucleotide sequence to the specific sequence) is determined by subtracting the distance from the labeled site of the target fragment to the specific sequence from the size (full-length) of the target fragment (distance from the known nucleotide sequence to the labeled end). The fragment size measurement is judiciously carried out by electrophoresis. Other measurement methods, such as mass spectrometry, are also available.

(2) Alternatively, a plurality of aliquots of the target fragment are prepared, and the target fragment aliquots are treated for cleavage at different specific sequences. When two different cleavage treatments are to be carried out, two aliquots of the target fragment are prepared, and one aliquot of the target fragment is cleaved at a first specific sequence to give first cleavage fragments. The other aliquot of the target fragment is cleaved at a second specific sequence to give second cleavage fragments. Of course, there may be present three or more specific sequences, and there may be produced three or more cleavage fragments. The respective cleavage fragments are subjected to the size measurement, and the predicted gene(s) is/are extracted from a database utilizing the sizes of the respective cleavage fragments, together with the respective specific sequences. When, in this manner, a plurality of cleavages are carried out, a plurality of fragments is obtained and, as a result, the probability of prediction of the gene increases or it becomes easy to identify a novel gene or useful gene.

(3) When the target fragment includes a known nucleotide sequence or a PCR product-derived fragment, it is efficient to carry out a homology search for the primer sequences using an existing gene database. As a primary search, a homology search is conducted for the known nucleotide sequences in the target fragment using an existing gene database, and genes having a sequence showing a level of homology not lower than a predetermined level are extracted from the gene database and the primary candidate database is thus obtained. In a secondary search, the predicted gene(s) is(are) extracted from the primary candidate database, utilizing the specific sequence (s) and the size(s) from the known nucleotide sequence used in the primary search to the specific sequence(s). Thus, the number of genes serving as candidates for the target fragment is reduced by carrying out a primary search through a database, in which a huge mass of genetic information is registered, using the known nucleotide sequence and further carrying out a secondary search using the information concerning the specific sequence(s) and the size from the known nucleotide sequence to the specific sequence(s). In this case, prior to conducting a search in a gene database using information of those fragments obtained by restriction enzyme treatment, a search concerning the known nucleotide sequence is conducted in a gene database, so that the number of candidates can be decreased to some extent in this stage of the search concerning the known nucleotide sequence, hence prediction can be conducted efficiently.

(4) It is recommended to provide the information of candidates for the target fragment in the form of a list. By presenting, in list format, the information for specifying a novel gene or useful gene, together with the specific sequence information about those genes, the database utilized and so forth, the useful information of candidates for the target gene can be utilized with ease, which is convenient for the user.

(5) Further, the novel gene or useful gene obtained as mentioned above in (1) to (4) may be cloned, followed by steps after acquisition of the novel gene or useful gene.

That is, by expressing a protein encoded by the cloned gene or gene fragment, and studying the behavior of the protein in cells, it becomes possible to identify the function of the gene. When the function of the gene becomes clear, the possibility of drug development, disease treatment, or biological phenomenon identification may arise.

By examining the upstream region of the cloned gene, it may become possible to identify the interaction with another protein and the function of the protein involved in the regulation of the expression of that gene. By studying the functions of proteins found successively, it may become possible to identify the signal transduction in cells stimulated by a signal factor such as a hormone or by an exogenous environmental factor, possibly leading to the identification of a biological phenomenon.

When the predicted gene is unknown or when the predicted gene is a fragment of an unidentified gene, the gene in question may be finally identified by expressing a protein encoded by the gene or gene fragment and studying the function of the protein.

(6) Further, the novel gene obtained by the above steps may be immobilized on a chip for utilization thereof in expression analysis through detection of a gene or genes capable of hybridizing with it. Alternatively, when it is a disease-related useful gene, the gene may be immobilized on a chip for utilization thereof in an expression evaluation system in gene diagnosis, for instance. Therefore, a method of producing chips with the immobilized novel gene(s) or useful gene(s) on chips is also provided. When a chip with this novel gene(s) or useful gene(s) immobilized thereon is subjected to hybridization using a solution of a nucleic acid probe having a sequence complementary to the novel gene(s) or useful gene(s), it is possible to evaluate the in vivo expression of a gene or a group of genes. Thus, when, in preparing a probe solution for hybridization, nucleic acid samples are prepared from two or more biological samples and labeled differently by origin, it is possible to simultaneously compare the levels of in vivo expression of the genes. Specifically, the chip with such gene(s) immobilized thereon can be applied in various types of gene diagnosis, for instance.

In the prior art, it is very time-consuming to identify, one by one, the DNA fragments obtained as a result of, or in the process of, an analysis. On the contrary, according to the present invention as described above, gene expression profiling can be conducted in a simple and easy manner and in a short period of time. Therefore, the present invention is useful in the sciences involved in new drug development as well as in the identification of various biological phenomena and activities.

In JP-A No. 2001-155035, it is described that a nucleotide sequence (tag) having a limited site in mRNA should be compared with a nucleotide sequence database. This publication describes preparing a DNA molecule comprising a plurality of tags by connecting short nucleotide sequence tags, and sequencing this DNA molecule to analyze transcription products successively and efficiently. On the other hand, the invention in the instant application comprises conducting a search in a gene database based on fragment size and a specific sequence(s) to thereby efficiently identify a useful gene or novel gene and, hence, is fundamentally different in object and search method from the invention in JP-A No. 2001-155035.

Other and further objects, features and advantages of the present invention will appear more fully from the following detailed description.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIGS. 1A to 1D show an example of cleavage of a target fragment with restriction enzymes, in which

FIG. 1A is a schematic representation of a target fragment and specific sites thereof,

FIG. 1B is a schematic representation of the corresponding methods of cleavage,

FIG. 1C is a schematic representation of images obtained by electrophoresis and visualization of cleavage fragments, and

FIG. 1D is a representation of the size information about the respective cleavage fragments;

FIG. 2 is a flowchart illustrating the process of prediction using fragment-predicting software;

FIG. 3 is a presentation of the results of electrophoresis following restriction enzyme cleavage;

FIG. 4 is a flowchart illustrating the method of searching using a known nucleotide sequence;

FIGS. 5A and 5B show the procedural flow of a gene prediction analysis according to the present invention, in which

FIG. 5A is a flowchart illustrating the procedural process of a gene prediction analysis, and

FIG. 5B is a flowchart illustrating the flow of an analysis using fragment-predicting software; and

FIG. 6 is a presentation of the result of an expression analysis using a DNA chip.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, other elements that may be well known. Those of ordinary skill in the art will recognize that other elements are desirable and/or required in order to implement the present invention. However, because such elements are well known in the art, and because they do not facilitate a better understanding of the present invention, a discussion of such elements is not provided herein. The detailed description will be provided herein below with reference to the attached drawings.

(Method of Preparing Nucleic Acid Samples)

In a first preferred embodiment of the present invention, a nucleic acid sample prepared from a biological sample.

The term “nucleic acid sample” means a mixture of mRNAs (messenger RNAs), total RNA containing mRNAs, or cDNAs (complementary DNAs) synthesized based on mRNAs, and DNAs, derived from a plurality of genes as extracted from a biological sample, such as cells, a tissue or tissues.

As for the method of preparation, methods known in the art can be applied according to various biological samples. For example, RNA can be purified by the AGPC method (Chomoczynski, P., and Sacchi, N. (1987), Anal. Biochem., 162, 156-159), for instance, and such a commercial reagent as TRIZOL Reagent (GIBCO BRL) can be used.

For gene amplification based on the nucleic acid sample prepared in the above manner, first strand cDNA synthesis is carried out based on the mRNA, namely the nucleic acid sample. The first strand cDNA synthesis can be carried out, for example, according to the technique of Sambrook, J. et al. (Molecular Cloning: A Laboratory Manual, 2nd ed., Cold Spring Harbor Laboratory Press), using the commercial kit SuperScript™ First-strand Synthesis System for RT-PCR (GIBCO BRL).

A plurality of fragments is thus obtained, which are then amplified. In recent years, various methods have been developed for gene amplification. In practicing the present invention, gene amplification can be carried out by utilizing PCR, among others.

(Target Fragment Selection)

Various methods are available for obtaining a target fragment according to the present invention. As a first example, the FDD method is preferably used for analyzing cDNA. The PCR method using a primer having a known nucleotide sequence is described by way of example. PCR is carried out using the above-mentioned first strand cDNA, together with a primer having a known nucleotide sequence and a terminally labeled oligo-d(T) primer. For making it possible to amplify a plurality of fragments simultaneously and carry out a plurality of reactions simultaneously in carrying out PCR, the above primer whose sequence is known is designed so that the size may be short, namely about 10 to 12 nucleotides (bases), and the GC content in the sequence may amount to about 50% to attain a uniform Tm (melting temperature). For example, primers attached to commercial DD kits (available from GenHunter Corporation or Clontech) or arbitrary primers (Welsh, J., and McClelland, M. (1990) Nucleic Acids Res., 18, 7213-7218) can be used.

As a result of PCR, a DNA solution containing a plurality of fragment species having the known nucleotide sequence at one end and a labeled sequence at the other can be prepared.

As a second example, there may be mentioned the AFLP method (Vos, P. et al., (1995), Nucleic Acids Res., 23, 4407-4414) for analyzing DNA. Genomic DNA is extracted from a material to be subjected to comparison. The genomic DNA prepared is sequence-specifically cleaved at a specific nucleotide sequence site. A synthetic oligonucleotide or a DNA sequence having a known nucleotide sequence, which is adapted to the cleavage fragments, is joined to the cleavage fragments. Then, PCR is carried out using a primer corresponding to the synthetic oligonucleotide or the above known nucleotide sequence. By this procedure, it is possible to amplify various fragments differing in size as resulting from sequence-specific cleavage at each specific nucleotide sequence site and thus obtain a plurality of gene fragments. This method can analyze cDNA as well (Hyeon-Se, L., and Jeffrey, C. Z., (2001), Proc. Natl. Acad. Sci. U.S.A., 98, 6753-6758).

In a third example, use can be made of DNA fragments introduced into a vector or phage by the shotgun cloning method (Sambrook, J. et al., (1989), Molecular Cloning: A Laboratory Manual, 2nd ed., Cold Spring Harbor Lab.) or the like. For example, when a library constructed by introducing fragments obtained by genome cleavage with a DNA-cleaving enzyme or the like into an appropriate vector is used as a nucleic acid sample, primers corresponding to sequences existing in the vector are designed and PCR is carried out using them, whereby a plurality of gene fragments can be obtained.

Furthermore, a plurality of fragments obtained by RACE may also be utilized. As a reference to the RACE method, there may be mentioned in Chenchik, A. et al., (1998), In TR-PCR methods for gene cloning and analysis. BioTechniques Books, MA, pp. 305-319.

It is noted that the above-mentioned genes or gene fragments need not always have a complete protein-encoding region. The reason is that the present invention uses, as targets of a search, those known and unknown gene DNA fragments registered in public databases and other databases as well.

(Target Fragment Labeling)

When PCR is used in the process of target fragment preparation, the target fragments can be labeled at their terminus by preliminarily labeling the primers. As for the labeling method, fluorescent-labeling, RI-labeling, labeling by biotinylation, or an equivalent method of labeling can be used. Further, an arrangement may be made for enabling secondary labeling of fragments instead of direct labeling thereof. Mostly, these methods of labeling can be carried out with ease by using commercial kits or entrusting with manufacturers engaged in synthesis services.

(Target Fragment Preparation)

When a target fragment is included among a plurality of DNA fragments, the target fragment alone is purified from the plurality of fragments. Electrophoresis is the simplest method of purification In the FDD or AFLP method mentioned above, an acrylamide gel is used as a means of analysis and therefore the target fragment can be excised from the analytical gel for purification. The gel excised is subjected to repetitions of freezing and thawing in purified water or an appropriate buffer solution for destructing of the gel, whereby the target fragment can be extracted. Then, PCR is carried out using the extracted DNA as a template to thereby amplify the target fragment again. The target fragment thus can be prepared in large amounts.

(Target Fragment Cleavage Treatment)

The sequence-specific cleavage treatment of the target fragment can be carried out in the following manner. The easiest and simplest method involves the use of a restriction enzyme(s) capable of cleaving DNA in a nucleotide sequence-specific manner (Sambrook, J., et al., (1989), Molecular Cloning 1: A Laboratory Manual, 2nd ed., Cold Spring Harbor Laboratory Press, 5.3-5.9). Which enzyme is to be used depends on the sequence and the size of the target fragment. Generally, the use of an enzyme recognizing a 4-base is recommended when the target fragment is not more than 1,000 bases in size. Of course, the use of an enzyme recognizing a 6-base is also effective but is not efficient since the probability of the specific sequence consisting of 6 bases being included in the target fragment sequence is low. Some restriction enzymes recognize a plurality of sequences. However, in principle, the use of such enzymes should be avoided since the search results become complicated. As other means for cleaving nucleic acids, artificial enzymes, typically ribozymes, or certain chemical substances can be used. An example of using a ribozyme (Einvik, C. et al., (1998), RNA 4: 530-541) is described below. A ribozyme can recognize and cleave a specific nucleotide sequence existing in RNA. By synthesizing cDNAs based on the RNA fragments obtained by cleavage, it is possible to obtain a plurality of fragments resulting from cleavage of the target fragment.

In carrying out sequence-specific cleavage, a specific sequence whose existence in the target fragment is infrequent is selected as the specific sequence to be cleaved so that the gene fragment is not, as far as is possible, cleaved at a plurality of sites. The reason for avoiding cleavage at a plurality of sites is that excessive shortening of the fragment size after cleavage, which makes the analysis difficult, should be avoided.

(Cleavage Fragment Size Measurement)

Methods of size measurement of the genes or gene fragments obtained by various methods are described below. Which method is to be used depends on the fragment sizes. When the fragment size is 1,000 bases or less, acrylamide gel electrophoresis or agarose electrophoresis is recommended. Acrylamide gel electrophoresis has a resolving power such that the difference in one base can be detected. It consumes time for analysis, however. Agarose gel electrophoresis is simple but the resolution is not so high. MALDI-TOF mass spectrometry can be carried out at high levels of resolution and throughput. When the target fragment is large, agarose electrophoresis is recommended. The size measurement by electrophoresis can be carried out by subjecting, simultaneously, DNA fragments (size markers) whose sizes are known to electrophoresis and comparing the mobility of a target fragment with those of size markers. This can be done in a simple and easy manner by using a sequencer or the like as the apparatus for electrophoresis.

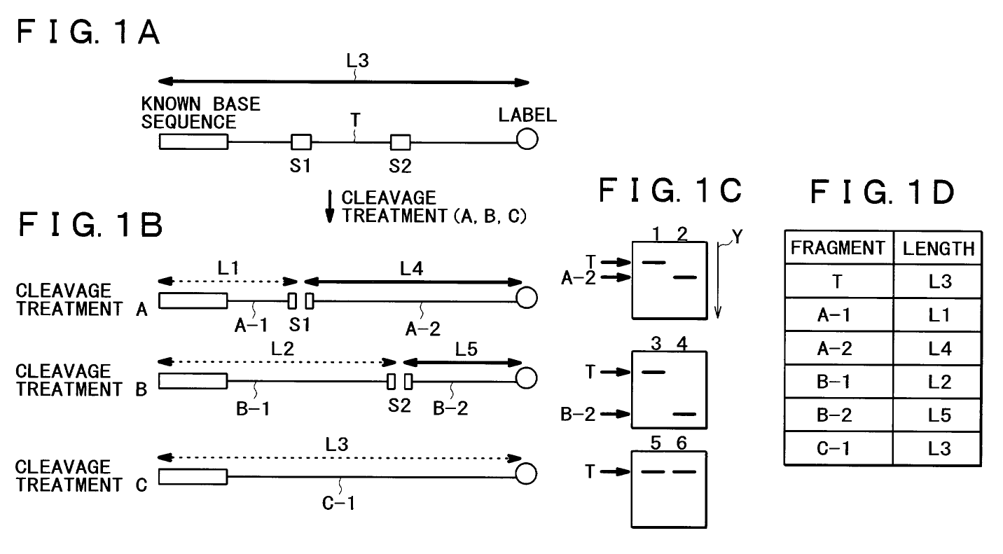

Taking the FDD method, whose analysis uses acrylamide gel electrophoresis, as an example, the size measurement of cleaved fragments is described referring to FIG. 1. Since the FDD method is a cDNA fingerprinting technology using a known nucleotide sequence and a fluorescent-labeled oligo-d(T) primer, the target fragment is obtained as a fragment having the known nucleotide sequence at one end and fluorescent-labeled at the other, as shown in FIG. 1A. Here, the results of treatment with three enzymes A, B and C differing in recognition and cleavage sequence are shown. The target fragment T has the size of L3 and has, in the sequence thereof, S1 and S2 cleavage sites recognized by the restriction enzymes A and B. When the target fragment is cleaved with the restriction enzyme A at the site S1, two fragments (A-1 and A-2) occur in the cleavage reaction mixture, and the fragment on the labeled side can be visualized by conducting electrophoresis using a fluorescent DNA sequencer. The image obtainable is shown in FIG. 1C. In lane 1, the target fragment (T) is detected and, in lane 2, a cleavage fragment (A-2) is detected, and the size information (L4) of fragment A-2 is obtained by the size measurement in comparison with size markers (not shown) simultaneously subjected to electrophoresis. In the same manner, two fragments (B-1 and B-2) obtained upon cleavage at the site S2 by the restriction enzyme B are electrophoresed in lane 4, and the size information (L5) of the fragment B-2 is obtained by the size measurement thereof. Further, when visualization of the labeled fragment after cleavage treatment C reveals only the same size as that of the target fragment, this means that the cleavage site for cleavage treatment C is absent in the target fragment. The size information of the respective cleavage fragments is summarized in FIG. 1D.

The process mentioned above includes the preparatory steps, inclusive of the target fragment preparation by the FDD method, prior to searching. When the labeled site and known nucleotide sequence are at one and the same end, the size of the cleavage fragment visualized among a plurality of the obtained fragments directly gives such information. The present invention can be applied also to the case where the target fragment is not labeled. A method of acquiring information without labeling is described below. First, the target fragment is cleaved at a plurality of specific sequences. When a restriction enzyme(s) is (are) used, for instance, the target fragment is cleaved with each restriction enzyme or a combination of a plurality of restriction enzymes. Then, the cleavage fragments are stained with a DNA staining agent, such as ethidium bromide, and the respective sizes are measured, whereby a physical map showing the sites of the specific sequences in the target fragment can be drawn. Utilizing this, information about the distances from the known nucleotide sequence to the specific sequences can be obtained instantaneously.

(Database)

It is preferable that the database used for the searching should be comprehensive and less redundant. Since the origin of the sample is known in most cases, the search can be efficiently conducted when the database used is species-specific. For example, UniGene of the National Center for Biotechnology Information (NCBI) belonging to the United States National Institute of Health (NIH) can be utilized. In addition, other genome databases, or databases not yet published but compiled by private enterprises through their own sequencing efforts, for instance, can also be utilized on the case-by-case basis. Furthermore, by consulting with a plurality of databases, not with one single database, it becomes possible to attain higher effectiveness and comprehensiveness.

(Target Fragment Prediction)

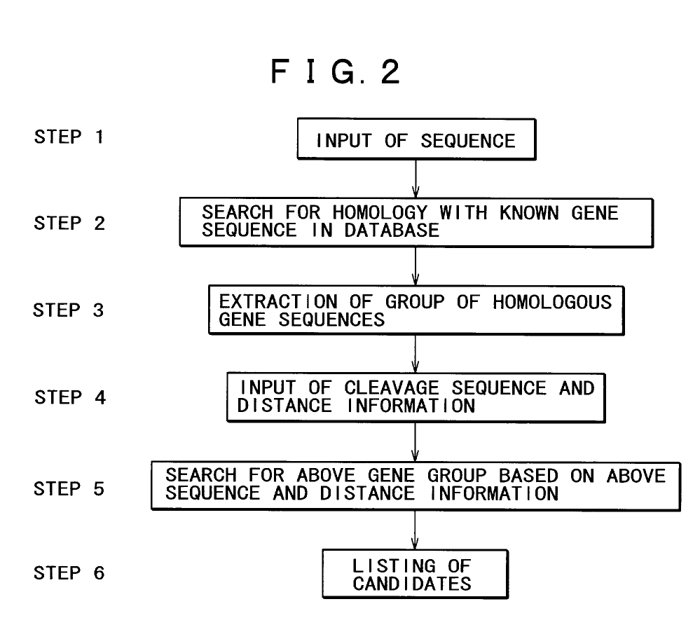

The flow of prediction for the target fragment is shown in FIG. 2 (flowchart). Here, a method of searching for the fragments having a known nucleotide sequence as obtained by the PCR method is described. Information concerning the genes showing homology to the known nucleotide sequence is extracted from the database employed for the search.

First, the known nucleotide sequence in the target fragment is inputted into fragment-predicting software. Based on the sequence, a homology search (primary search) is conducted in a known gene database, and a group of nucleotide sequences having homology is extracted. For conducting the search more efficiently on this occasion, it is preferable that a reference value for the search be selected so that not only a group of genes showing 100% homology but also a group of genes showing a high level of homology exceeding this reference value are extracted. In conducting a search for a fragment obtained as a result of PCR, homology search seems to be most effective, when conducted by differently giving added weight to the respective bases in the primer sequence, which is the known nucleotide sequence. That is, each base at each position is not always a coinciding base. In the FDD method, for instance, PCR is carried out under relatively mild conditions using primers of about 10 bases in size, so that the target fragment amplified does not always have 100% homology to such primer sequence. However, our analysis has revealed that a portion closer to the 3′ end of the primer sequence has approximately 100% homology and that, conversely, differences by 2 bases, if any, are mostly found in the 5′ end portion. More specifically, by setting the cut-off value (score value) at 90 and allocating the weighting values 5′ 5/5/5/5/10/10/10/10/20/20 3′ to the respective position of the base in the primer sequence, a search is conducted in a manner such that a group of homologous genes possibly containing one or two mismatched bases in each of the 4 bases from the 5′ end of the known nucleotide sequence (in this case 10 bases), one mismatched base in each of the 5th to 8th base and no mismatched base in the 2 bases at the 3′ end may be extracted. The score value and weighting values are preferably varied according to the method of acquisition of the target fragment. Further, by selecting a plurality of search conditions for one and the same target fragment, it also becomes possible to obtain search results differing in reliability.

The gene group found is stored as a primary candidate database. Then, the information obtained by cleavage treatment of the target fragment is inputted in the form of specific cleavage sequence and cleavage fragment size, and a search is conducted for those nucleotide sequences in which the above specific sequence occurs at a site corresponding to the cleavage fragment size from among the primary candidate database. It is effective to conduct a closer search by carrying out a plurality of cleavage treatments. In the case of cleavage with a plurality of restriction enzymes as mentioned hereinabove referring to FIG. 1, for instance, the target fragment T having a known nucleotide sequence at one end and labeled at the other end is separately treated with three different restriction enzymes, followed by electrophoresis, which gives three labeled fragments, A-2, B-2 and C-1. The sites of the restriction enzyme recognition sequences relative to the known nucleotide sequence can easily be determined by subtracting the fragment size, for example L4 for A-2, from the L3 size of the fragment T. For conducting a closer search, it is effective to input such information according to a logical equation. In the case of FIG. 1, nucleotide sequences which satisfy the logic “enzyme treatment A” AND “enzyme treatment B” NOT “enzyme treatment C” are searched for. The enzyme treatment C provides information stating that the target fragment was not cleaved by that treatment, namely it has no relevant recognition sequence; this information can effectively be used as information for conducting a search.

The predicted genes, which are obtained from those searches, are preferably provided in the form of a list. When the search conditions are not enough but the number of candidate genes is great, the list may serve as a guide in conducting a further cleavage treatment. However, when there is no gene of interest in the list, the analysis may be discontinued. In certain cases, the search is enough and one single gene may be identified. Conversely, in other cases, there may be no candidate gene since the gene in question is not yet contained in any existing database. The present invention makes it possible to predict in advance that the gene in question may be a novel one. The present invention thus makes it possible to carry out detailed gene analysis, such as sequencing, with efficiency, so that the study program as a whole can be expedited and the cost can be reduced.

(Utilization of Search Results)

In accordance with the present invention, the conventional methods can be carried out speedily and inexpensively. When, however, preferred methods of the present invention are practiced as described below, a more effective analysis can be made.

Protein analysis is indispensable for closely investigating the function of a gene. The above search results give information about the gene from which the target fragment is derived. Therefore, based on the information from the database, primers are designed so that they may cover the full-length of the gene. It is easy to isolate the full-length of the gene from the starting material, in which the target fragment is contained, by the PCR method using the above primers.

It is also possible to carry out a DNA chip analysis utilizing the nucleotide sequences of candidate genes. In most of commercial chips, known genes or ESTs are immobilized on a substrate. Those chips are not always suited for targets of analysis. However, to develop chips adapted to targets of analysis is a costly and time- and labor-consuming work. By applying the present invention, it is possible to provide highly efficient chips at relatively low cost. The FDD method and other cDNA fingerprinting technologies can analyze a target of analysis comprehensively but at the same time have problems; for example, no gene information can be obtained until a target fragment is cloned and sequenced. In accordance with the present invention, however, it is now possible to immobilize, on a chip, a group of genes of interest as selected by preliminary screening through the present invention. It is thus possible to make an inclusive chip adapted to a target of analysis with great efficiency. In DNA chip making, it is important to pay attention to the following.

For carrying out hybridization between each fragment immobilized on a DNA chip and a sample-derived DNA fragment under highly accurate (or highly stringent) conditions, the relation between hybridization temperature and the melting temperature (Tm) of the immobilized fragment is important and it is necessary that the difference between the Tm of the immobilized DNA fragment and the hybridization temperature should not exceed 30° C. Further, for preventing cross-hybridization, it is necessary that the homology between the immobilized DNA fragment and a DNA fragment, among sample-derived DNA fragments, nonhybridizing in itself with the immobilized DNA fragment should be sufficiently low. Furthermore, it is desirable that any portion highly homologous to a sequence building a minihairpin structure or to a repetitive sequence known as Alu sequence in the case of human genes be not contained.

Each DNA fragment or oligonucleotide to be immobilized and meeting the above conditions is adjusted to an appropriate concentration (0.1 to 1.0 μg/l), and then spotted onto a slide glass coated in advance with polylysine or an aminosilane using a spotter. The DNA fragment or oligonucleotide can thereby be immobilized on the chip.

Cy5-labeled cDNA is synthesized by the reverse transcription reaction using mRNA of one sample and Cy5-dCTP, while Cy3-labeled cDNA is synthesized by the reverse transcription reaction using mRNA of the other sample and Cy3-dCTP. A mixed solution composed of equal amounts of these Cy5-labeled cDNA and Cy3-labeled cDNA is submitted to hybridization. The hybridization temperature is preferably 45 to 70° C., and the hybridization time 6 to 18 hours. After hybridization, the fluorescence intensities respectively due to Cy5 and Cy3 at the site of spotting of each gene are measured using a fluorescence scanner, whereby the difference in expression level between both can be determined.

The following preferred examples further illustrate the present invention.

### EXAMPLE 1

The FDD method was employed for selecting target fragments from among a plurality of gene fragments obtained. The FDD method is a method for gene expression analysis which comprises comparing, between samples, cDNA fingerprints obtained by using arbitrary sequences. This method is an excellent method of comprehensively analyzing a large number of expressed genes by using a large number of primers. It is necessary, however, to isolate and identify gene fragments of interest from the fingerprints.

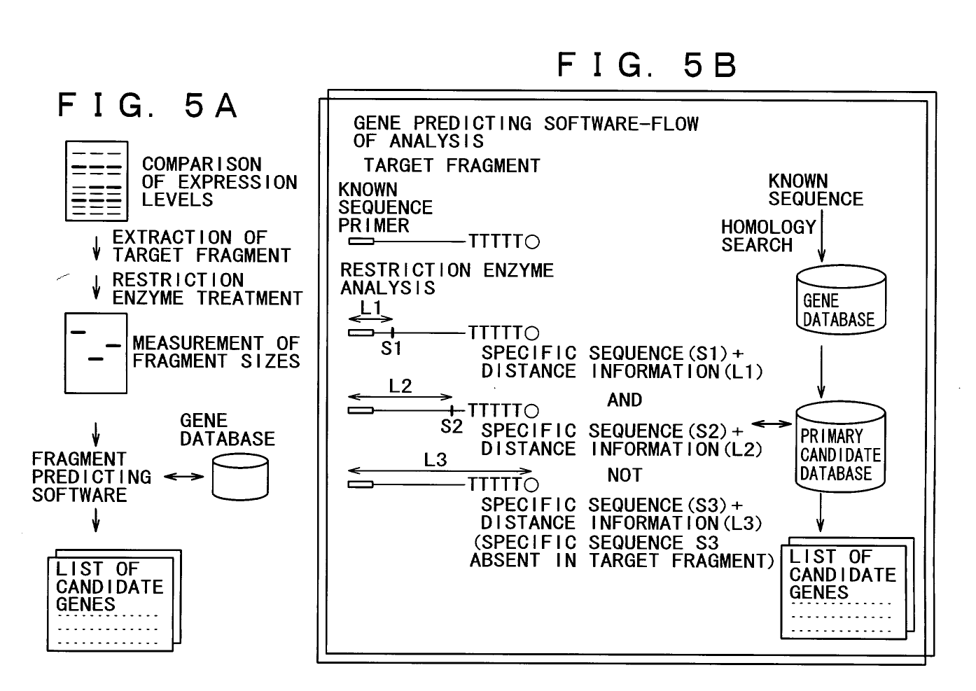

Now, referring to FIG. 5, an analytical method for predicting and identifying gene fragments utilizing the FDD method is described in detail. FIG. 5A illustrates the flow for preparing target fragments, and FIG. 5B illustrates the flow of the method of analysis.

A. Target Fragment Preparation

Prior to target fragment preparation, first strand cDNA, namely the origin of target fragments, was synthesized from mouse mRNA. The synthesis was carried out using oligo-d(T) primer (derived from poly(T) sequence by addition of G to the 3′ end thereof) and the commercial kit SuperScript™ First-strand Synthesis System for RT-PCR (GIBCO BRL). Then, with the thus-synthesized first strand cDNA as the template, PCR was carried out using oligo-d(T) primer labeled with the fluorescent Texas Red and a primer having a known nucleotide sequence. The sequence of the primer for PCR was selected so that the following conditions might be satisfied: the primers should not pair with each other; the sequence selected should hardly form an intramolecular hydrogen bond; the Tm of the primer and gene is desirably within the appropriate range (40 to 70° C.). The respective primers are shown below.

Oligo-d(T) primer (Nippon Flour Mills) : SEQ ID NO:3 in the sequence listing

Known nucleotide sequence primer (OPERON TECHNOLOGY, Inc.): SEQ ID NO:1 or 2 in the sequence listing

PCR was carried out using the combination (1) of the known nucleotide sequence primer (SEQ ID NO:1) and the fluorescent-labeled oligo-d(T) primer, and the combination (2) of the known nucleotide sequence primer (SEQ ID NO:2) and the fluorescent-labeled oligo-d(T) primer.

The PCR reaction mixture was prepared according to Takamichi Muramatsu's method of FDD (Shokubutsu no PCR Jikken Purotokoru (Protocols for PCR Experiments in Plants), New Edition, 138-143, published by Shujunsha), as follows. The composition of the mixture is shown in Table 1.

TABLE 1
<Composition of the Mixture>

| | |
|---|---|
| 2.5 mM dNTP (Nippon Gene) | 0.4 μl |
| 10 × GeneTaq buffer (Nippon Gene) | 2.0 μl |
| 1.0 μM oligo-d(T) primer | 5.0 μl |
| 10 μM primer whose sequence is known | 1.0 μl |
| GeneTaq (Nippon Gene) | 0.1 μl |
| AmpliTaq (Perkin Elmer) | 0.1 μl |
| purified water | 10.4 μl |
| Total | 19.0 μl |

1. Reaction Method

A PCR reaction mixture (20.0 μl) was prepared by adding 19.0 μl of the mixture shown in Table 1 to 1.0 μl of the first strand cDNA. As for the temperature cycling in PCR, the first cycle was carried out at 94° C. for 3 minutes, then at 40° C. for 5 minutes, and at 72° C. for 5 minutes, the succeeding 24 cycles were each carried out at 94° C. for 20 seconds, at 40° C. for 2 minutes and at 72° C. for 1 minute, and then the reaction was further carried out at 72° C. for 5 minutes.

2. PCR Product Detection

To a 2.0-μl-portion of the PCR product was added an equal volume of loading buffer (98% formamide, 1.0 mM EDTA, 0.01% Methyl Violet), followed by 2 minutes of treatment at 80° C., to give a sample for electrophoresis.

Electrophoresis was carried out using a Hitachi DNA sequencer under acrylamide gel (6% LongRanger (Takara Shuzo), 6.1 M urea, 1.2×TBE) conditions for separation of sample DNAs and molecular weight markers (100-base-ladder markers from 100 bases to 1,000 bases). Then, DNA band detection was carried out using a fluorescent image scanner (Hitachi software FMBIO).

From among the fragments obtained by carrying out the PCR using the primer combination (1), one was selected and designated as target fragment 1. In the same manner, one was selected from among the PCR products obtained with the combination (2) and designated as target fragment 2. Comparison of the mobilities of the target fragments with those of the molecular weight markers revealed that the target fragment 1 was 795 bp long and the target fragment 2 430 bp long.

3. Target Fragment Excision and Reamplification

Each target fragment gel fraction was excised from the gel and placed in a 1.5-ml tube. Purified water (30.0 μl) was added to the tube, which was then allowed to stand in a freezer at −80° C. for 10 minutes. The tube was then taken out, and the sample was dissolved by stirring with a vortex mixer at room temperature for 10 minutes. This procedure was repeated twice for DNA extraction. Then, using 1.0 μl of each extract as a template, the target fragment was amplified under the same PCR conditions as mentioned above.

B. Fragment Cutting and Size Measurement

1. Restriction Enzyme Treatment

Each reamplified target fragment is cleaved with a plurality of restriction enzymes. Those restriction enzymes for which the number of recognition sequences (specific sequences) on the gene is considered to be relatively small are selected as the restriction enzymes to be used so that, if possible, the DNA fragment may not be cleaved at a plurality of sites. In the practice of the present invention, the restriction enzymes Hae III, Sau3A I and Taq I, which recognize and cleave the specific 4-base, were used.

2. Confirmation by Electrophoresis and Size Measurement of Cleavage Fragments

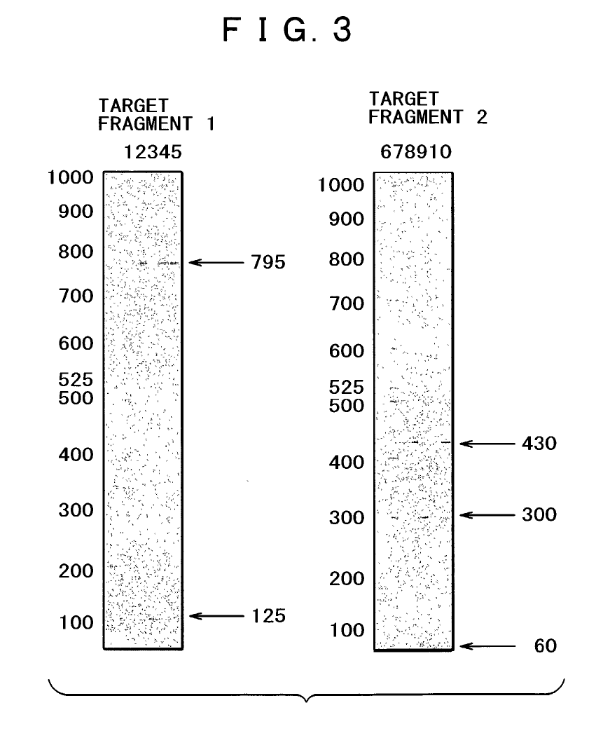

The plurality of DNA fragments obtained by restriction enzyme cleavage were separated by electrophoresis using a Hitachi DNA sequencer, and the fragments on the labeled side were visualized. The results are shown in FIG. 3 as the results of electrophoresis following restriction enzyme cleavage. The figures of 100 to 1,000 corresponding to the DNA bands in lanes 1 and 6 indicate the size (bp) of size markers. In lane 2, the untreated target fragment 1 DNA was electrophoresed; the size of the target fragment 1 is 795 bp. In lanes 3, 4 and 5, the products of cleavage treatment of target fragment 1 with the restriction enzymes Hae III, Sau3A I, and Taq I, respectively, were electrophoresed. As a result of cleavage treatment with the restriction enzyme Hae III, a 125-bp cleavage fragment was obtained, and this revealed that there is a Hae III cleavage site in the nucleotide sequence of the gene contained in target fragment 1. On the other hand, treatment with the restriction enzyme Sau3A I or Taq I gave a fragment having the same size as the size of the starting target fragment 1, whereby there is no cleavage site for either of these enzymes.

In lane 7, the untreated target fragment 2 DNA was electrophoresed; the size of the target fragment 2 is 430 bp. In lanes 8, 9 and 10, the products of cleavage treatment of target fragment 2 with the restriction enzymes Hae III, Sau3A I, and Taq I, respectively, were electrophoresed. Treatment with the restriction enzyme Hae III gave a 300-bp cleavage fragment and treatment with the restriction enzyme Sau3A I gave a 60-bp cleavage fragment, revealing that there are cleavage sites for these enzymes in target fragment 2. On the other hand, treatment with the restriction enzyme Taq I gave a fragment having the same size as the size of the untreated target fragment 2, whereby it was revealed that there is no cleavage site for this enzyme.

Since the label was on the oligo-d(T) primer side, it is necessary to calculate the size from the known nucleotide sequence to the specific cleavage site such as the restriction enzyme recognition sequence (specific sequence). This can easily be obtained by subtracting the size of the DNA fragment after enzyme treatment as obtained in the above manner from the size of the target fragment. The results obtained in the above manner are summarized in Table 2 and Table 3.

TABLE 2
<Sizes of the target fragments after cleavage>

| | | Fragment size after | | |
|---|---|---|---|---|
| | Size | enzyme treatment | | |
| Target | (Full-length) | Hae III | Sau3 AI | Taq I |
| fragment | (bp) | (GG!CC) | (!GATC) | (T!CGA) |
| Target | 795 | 125 | 795 | 795 |
| fragment | | | No clea- | No cleav- |
| 1 | | | vage site | age site |
| Target | 430 | 300 | 60 | 430 |
| fragment | | | | No cleav- |
| 2 | | | | age site |

!is the site actually cleaved

TABLE 3
<Sizes used for actual search>

| | | Value obtained by subtracting | | |
|---|---|---|---|---|
| | | fragment size after enzyme | | |
| | Size | treatment from the size of | | |
| Target | (Full-length) | the target fragment | | |
| fragment | (bp) | Hae III | Sau3 AI | Taq I |
| Target | 795 | 670 | 0 | 0 |
| fragment | | | | |
| 1 | | | | |
| Target | 430 | 130 | 370 | 0 |
| fragment | | | | |
| 2 | | | | |

C. Search Method

C-1. Primary Search Method

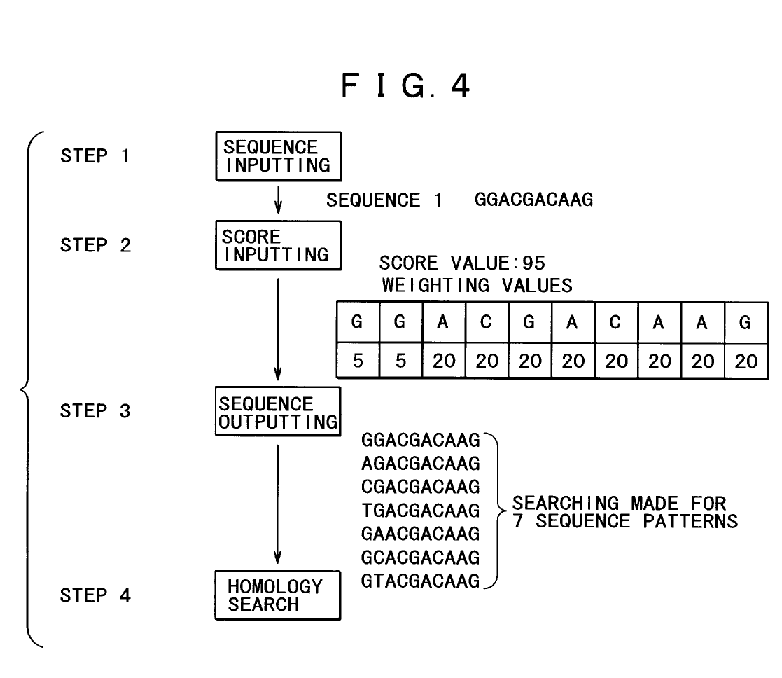

For investigating the efficacy of the search method of the present invention, fragment-predicting software was tentatively prepared. The experiment mentioned below was carried out using this software. In this example, the NCBI UniGene mouse database was used as the database. For carrying out a verification experiment using a plurality of databases, two databases, Mm.seq.all and Mm.seq.uniq., were used. The known nucleotide sequence of a terminus of the target fragment (the primer sequence is used since, in this example, the target fragment is a PCR product) is inputted into the fragment-predicting software, and genes having homology to the above known nucleotide sequence were searched for among the database sequences (primary search). In the primary search, a closer search can be conducted by inputting a plurality of parameters. This is made considering the fact that the primers obtained do not always fully correspond to the primary sequence used. The flow from primer sequence inputting to database search is shown in FIG. 4 as a search method using a known nucleotide sequence. Referring to this figure, the primer sequence inputting method is explained. First, the primer sequence is inputted, and the proportion (score value) in which a base or bases not coinciding with the base or basses in the primer sequence are contained is inputted. Then, weighting values for the respective bases in the primer sequence, which mean that “the base in question is not always a coinciding base”, are inputted. For example, when the primer sequence is GGACGACAAG, 95 is employed as the score value, and 5′ 5/5/20/20/20/20/20/20/20/20 3+ is used for weighting the respective bases. The software automatically makes calculations and conducts a search in a manner such that the sum of the score value and the weighting value (in this case, 5) given to each base with the condition that “a base may be a non-coinciding one” amounts to 100. Thus, it means that a sequence which differs from the nucleotide sequence inputted with respect to either of the 5′-terminal GG but otherwise is fully coinciding is a target of the search. Therefore, in the above instance, the search is conducted for 7 sequence patterns.

The above search focuses on not only genes homologous to the known nucleotide sequence inputted but also genes complementary to the known nucleotide sequence. This is because the directionality of the known nucleotide sequence is unknown and because the directions of genes registered in the database are unknown.

In this verification experiment, the database Mm.seq.uniq. was used. In the Mm.seq.uniq. database, overlapping gene fragments have been excluded and, furthermore, treatment has been made to join individual gene fragments via an overlapping portion, if any. Thus, full-length genes or relatively long gene fragments are registered in that database. The software was loaded with this database, and searches were conducted under various conditions changing the score value and weighting values. As a result of searching under the conditions of complete agreement (score value: 100) with the nucleotide sequence inputted, 53 clones were hit. Further, when searching was conducted under the conditions that the terminal one or two bases might differ from those in the sequence inputted, 170 clones and 535 clones were hit, respectively. These search results were stored as a primary candidate database.

C-2. Secondary Search Method (Effect of Logical Equation)

Further, for narrowing the range of the primary candidate database obtained in the above manner, the restriction enzyme recognition sequence (specific sequence) and the size (Table 3) from the known nucleotide sequence to the restriction enzyme recognition sequence were inputted, and a search was again conducted on the primary candidate database. Here, for narrowing the range of the candidate list, a plurality of specific sequences and of sequence-to-sequence distances can be inputted. Further, each piece of information to be inputted can be accompanied by a logical equation given by using AND, OR and/or NOT and, thus, combined conditions can be given. Thus, when the fragment in question is cleaved with the restriction enzymes A and B but not with the restriction enzyme C, the primary candidate list can be narrowed by designating the information concerning the restriction enzymes A and B as AND while designating the information on the restriction enzyme C as NOT. OR may be used when it is not desired to specify the occurrence or nonoccurrence of cleavage. Since NOT thus means nonoccurrence of cleavage, the number of candidates can be reduced by conducting the search using the NOT information. As for the sequence-to-sequence distance information, the size measurement of the fragments resulting from restriction enzyme cleavage is not so accurate and, therefore, the range of values tolerable as errors was inputted for conducting a search.

Target fragment 1 was cleaved at a site of 125 bases from the labeled terminus with the restriction enzyme Hae III and, therefore, the restriction enzyme recognition sequence (specific sequence) GG!CC and the values 125±10 as the cleavage fragment size ± tolerance were inputted. Further, since the restriction enzymes Sau3A I and Taq I both failed to cause cleavage, the condition NOT was inputted, and a secondary search was conducted. The search conditions and results are shown in Table 4.

TABLE 4
<Search conditions for fragment 1 (database: Mm.seq.uniq.)>

| | | Primary search | | | | |
|---|---|---|---|---|---|---|
| | | results (number | | | | Secondary search |
| | | of primary | | | | results (number |
| | | candidates | Restriction enzyme-cleaved fragment size ± | | | of genes |
| | | registered in | tolerance | | | registered in |
| Score | Weighting values | database ) | (GG!CC) | (!GATC) | (T!CGA) | candidate list) |
| 100 | 20 20 20 20 20 20 20 20 20 20 | 53 | 125 ± 10 | Not | Not | 0 |
| 95 | 5 20 20 20 20 20 20 20 20 20 | 170 | 125 ± 10 | Not | Not | 0 |
| 90 | 5 5 20 20 20 20 20 20 20 20 | 535 | 125 ± 10 | Not | Not | 2 |
| | | | 125 ± 5 | Not | Not | 1 |
| | | | 125 ± 2 | Not | Not | 1 |
| | | | 125 ± 1 | Not | Not | 1 |
| | | | 125 ± 0 | Not | Not | 0 |

At score value: 100, no gene was hit in the primary candidate database. However, when a primary search was conducted at score value: 95 and a secondary search was conducted with a tolerance of ±10 with respect to the restriction enzyme information, 2 clones were hit. Under more strict conditions, namely when the tolerance was not more than ±5, one clone was hit.

### EXAMPLE 2

### Provision of a List

The secondary search results are stored as a candidate gene list. The candidate gene list, which is the result of searching conducted at score value: 90 and a tolerance of ±1 is shown in Table 5.

TABLE 5
<List for target fragment 1>

| Name of gene | Database used | UniGene No. | GenBank No. | Sequence | Library |
|---|---|---|---|---|---|
| Mus musculus integral | UniGene | Mm.4266 | NM 008410 | Sequence No. 4 | — |
| membrane protein 2B | (Mm.seq.uniq) | | | | |

The name of the candidate gene obtained was Mus musculus integral membrane protein 2B, with a UniGene number of Mm. 4266 (GenBank NM 008410), and the sequence thereof is shown in the sequence listing under SEQ ID NO:4.

### EXAMPLE 3

### Experiment for Verifying the Search Results

For checking as to whether the nucleotide sequence of target fragment 1 coincides with that of the gene predicted by the software, the gene contained in the target fragment was purified and cloned. Promega's pGEM-T vector was used as the vector. After concentration adjustment to vector:purified target fragment 1 gene=1:3 to 10, ligase buffer was added to make the whole amount 9.5 μl, 0.5 μl of ligase was further added, and ligation was carried out at room temperature for 30 minutes to cause the vector and target fragment to join to each other. The thus-obtained plasmid DNA was amplified in quantity in Escherichia coli, and purified using a commercial kit (BIO 101, RPM kit). The nucleotide sequence of the target fragment was determined using a sequencer (Perkin Elmer, ABI 377). The sequencing PCR reaction was carried out using Perkin Elmer's ABI PRISM dGTP BigDye Terminator Ready Reaction Kit. The nucleotide sequence of the gene as obtained by sequencing is shown in the sequence listing under SEQ ID NO:5.

Based on the nucleotide sequence obtained as a result of sequence analysis, a BLAST search was conducted, whereupon the sequence showed 99% homology to Integral membrane protein 2B. The target fragment 1 was thus found to be the known gene Integral membrane protein 2B, and the result obtained by using the fragment-predicting software was verified. Thus, the fragment-predicting software was proved to be a means for carrying out a rapid gene prediction in gene analysis.

### EXAMPLE 4

### The Case of a Novel Gene

Then, for target fragment 2, searches were conducted while varying the score value and weighting values in various ways, as shown in Table 6.

TABLE 6
<Search conditions for fragment 2 (database: Mm.seq.uniq.)>

| | | Primary search | | | | |
|---|---|---|---|---|---|---|
| | | results (number | | | | Secondary search |
| | | of primary | | | | results (number |
| | | candidates | Restriction enzyme-cleaved fragment size ± | | | of genes |
| | | registered in | tolerance | | | registered in |
| Score | Weighting values | database) | (GG!CC) | (!GATC) | (T!CGA) | candidate list) |
| 100 | 20 20 20 20 20 20 20 20 20 20 | 15 | 300 ± 10 | 60 ± 10 | Not | 0 |
| 90 | 5 5 20 20 20 20 20 20 20 20 | 460 | 300 ± 10 | 60 ± 10 | Not | 0 |
| 80 | 5 5 5 5 20 20 20 20 20 20 | 6143 | 300 ± 10 | 60 ± 10 | Not | 0 |
| 70 | 5 5 5 5 5 5 5 20 20 20 | 83605 | 300 ± 10 | 60 ± 10 | Not | 0 |
| 50 | 10 10 10 10 10 10 10 10 10 10 | 85119 | 300 ± 10 | 60 ± 10 | Not | 0 |
| 40 | 10 10 10 10 10 10 10 10 10 10 | 85122 | 300 ± 10 | 60 ± 10 | Not | 0 |

However, all the conditions inputted into the fragment-predicting software failed to find out any corresponding gene. The list obtained by the fragment-predicting software is shown in Table 7.

TABLE 7
<List for target fragment 2>

| Name of gene | Database used | UniGene No. | GenBank No. | Sequence | Library |
|---|---|---|---|---|---|
| Unknown | UniGene (Mm.seq.uniq) | — | NM 008410 | — | — |

Since no corresponding gene was found, cloning was carried out in the same manner as with the target fragment 1, and the nucleotide sequence thereof was determined using a sequencer. Then, using the nucleotide sequence thus revealed, an ordinary BLAST search was conducted. No homologous gene was found, hence the gene was found to be a novel one. When no corresponding gene is found in searching using the fragment-predicting software, the possibility is high of the gene being a novel one. Thus, it was revealed that the gene prediction method provided by the present invention is a very efficient and effective means for rapidly finding out a novel gene and efficiently cloning the same.

The nucleotide sequence of target fragment 2 is shown in the sequence listing under SEQ ID NO:6. Cloning of an unknown gene is a means for obtaining a full-size gene and, based on this information, it becomes possible to carry out a RACE experiment or a library screening method.

### EXAMPLE 5

### Searching Using a Different Database

Then, a verification experiment was carried out in the same manner using another database, namely Mm.seq.all. The conditions inputted are summarized in Table 8 and Table 9.

TABLE 8
<Search conditions for fragment 1 (database: Mm.seq.all)>

| | | Primary search | | | | |
|---|---|---|---|---|---|---|
| | | results (number | | | | Secondary search |
| | | of primary | | | | results (number |
| | | candidates | Restriction enzyme-cleaved fragment size ± | | | of genes |
| | | registered in | tolerance | | | registered in |
| Score | Weighting values | database) | (GG!CC) | (!GATC) | (T!CGA) | candidate list) |
| 100 | 20 20 20 20 20 20 20 20 20 20 | 705 | 125 ± 10 | Not | Not | 1 |
| 95 | 5 20 20 20 20 20 20 20 20 20 | 2292 | 125 ± 10 | Not | Not | 1 |
| 95 | 5 5 20 20 20 20 20 20 20 20 | 3342 | 125 ± 10 | Not | Not | 1 |
| 90 | 5 5 20 20 20 20 20 20 20 20 | 7719 | 125 ± 10 | Not | Not | 9(6) |
| | | | 125 ± 5 | Not | Not | 8(6) |
| | | | 125 ± 2 | Not | Not | 7(6) |
| | | | 125 ± 1 | Not | Not | 5(5) |
| | | | 125 ± 0 | Not | Not | 1(1) |

TABLE 9
<Search conditions for fragment 2 (database: Mm.seq.all)>

| | | Primary search | | | | |
|---|---|---|---|---|---|---|
| | | results (number | | | | Secondary search |
| | | of primary | | | | results (number |
| | | candidates | Restriction enzyme-cleaved fragment size ± | | | of genes |
| | | registered in | tolerance | | | registered in |
| Score | Weighting values | database) | (GG!CC) | (!GATC) | (T!CGA) | candidate list) |
| 100 | 20 20 20 20 20 20 20 20 20 20 | 142 | 300 ± 10 | 60 ± 10 | Not | 0 |
| 90 | 5 5 20 20 20 20 20 20 20 20 | 5628 | 300 ± 10 | 60 ± 10 | Not | 0 |
| 80 | 5 5 5 5 20 20 20 20 20 20 | 87146 | 300 ± 10 | 60 ± 10 | Not | 0 |
| 70 | 5 5 5 5 5 5 5 20 20 20 | 1852724 | 300 ± 10 | 60 ± 10 | Not | 0 |
| 50 | 10 10 10 10 10 10 10 10 10 10 | 1897497 | 300 ± 10 | 60 ± 10 | Not | 0 |
| 40 | 10 10 10 10 10 10 10 10 10 10 | 1897521 | 300 ± 10 | 60 ± 10 | Not | 0 |

Since overlapping gene fragments are registered in the Mm.seq.all database, a larger number of gene fragments were hit as compared with the search in Mm.seq.uniq. The figures in the parentheses in Table 8 each indicate the number of gene fragments shorter than or homologous to the nucleotide sequence of the clone Mm.4266 (GenBank NM 008410) (Mus musculus integral membrane protein 2B gene) as obtained in Example 2.

The lists obtained by the respective target fragment searches are shown in Table 10 and Table 11. In Table 10, the candidate genes for target fragment 1 are shown as a result of searching conducted at score value: 90 and a tolerance of ±1.

TABLE 10
<List for target fragment 1>

| Name of gene | Database used | UniGene No. | GenBank No. | Sequence | Library |
|---|---|---|---|---|---|
| Mus musculus | UniGene | Mm.4266 | NM 008410 | Sequence No. 4 | — |
| integral membrane | (Mm.seq.all) | | | | |
| protein 2B | | | | | |
| Mus musculus | UniGene | Mm.4266 | U76253 | Sequence No. 7 | cDNA library of the |
| integral membrane | (Mm.seq.all) | | | | osteogenic stromal |
| protein 2B | | | | | cell line MN7 |
| Mus musculus | UniGene | Mm.4266 | AB030203 | Sequence No. 8 | — |
| integral membrane | (Mm.seq.all) | | | | |
| protein 2B | | | | | |
| Mus musculus | UniGene | Mm.4266 | BC004731 | Sequence No. 9 | — |
| integral membrane | (Mm.seq.all) | | | | |
| protein 2B | | | | | |
| Mus musculus | UniGene | Mm.4266 | AK005125 | Sequence No. | Mus musculus adult |
| integral membrane | (Mm.seq.all) | | | 10 | male cDNA RIKEN |
| protein 2B | | | | | full-size enriched |
| | | | | | library |

TABLE 11
<List for target fragment 2>

| Name of gene | Database used | UniGene No. | GenBank No. | Sequence | Library |
|---|---|---|---|---|---|
| Unknown | UniGene (Mm.seq.all) | — | — | — | — |

Since the same gene was hit in the different databases, it was revealed that the fragment-predicting software is applicable to a plurality of databases.

### EXAMPLE 6

### Chip

1. Oligonucleotide Probe Designing

Then, the gene revealed by combining the FDD method with the present invention was immobilized on a DNA chip, a gene expression analysis was carried out using the DNA chip, and whether this could be applied to gene diagnosis or the like or not was checked. Based on the nucleotide sequences of the target fragments 1 and 2 obtained in the above-mentioned search, oligonucleotide probes were designed. In the designing, the longest sequence information obtained as a search result was used. For designing probe sequences suited for DNA chip analysis, the application software named Oligo (Molecular Biology Insights) was used. In determining which portion of the nucleotide sequence is to be used as a probe, care should be paid to the fact that the difference between the hybridization temperature and the melting point of the immobilized DNA fragment should not exceed 30° C. Oligonucleotide probes were designed based on the nucleotide sequences corresponding to target fragments 3 to 10 obtained in the same manner as in the preceding example, to increase the number of examples of analysis using the DNA chip. The list of target fragments 3 to 10 is shown in Table 12.

TABLE 12
<Genes used in DNA chip analysis>

| Fragment species | UniGene No. | Name of gene |
|---|---|---|
| Target | Mm.4266 | Mus musculus integral |
| fragment 1 | | membrane protein 2B |
| Target | — | unknown |
| fragment 2 | | |
| Target | Mm.21567 | Mus musculus intracellular |
| fragment 3 | | calcium-binding protein |
| Target | Mm.142188 | Cytochrome C oxidase |
| fragment 4 | | polypeptide VIIB precursor |
| Target | Mm.35439 | Cystein-rich glycoprotein |
| fragment 5 | | SPARC |
| Target | Mm.30028 | Mus musculus RIKEN cDNA |
| fragment 6 | | 1110014J03 gene |
| Target | Mm.1548 | Mus musculus |
| fragment 7 | | alpha-1,3-galactosyltransferase |
| Target | Mm.203803 | NCI_CGAP_SG2 Mus musculus |
| fragment 8 | | cDNA clone image: 4192740 |
| Target | Mm.192208 | Mus musculus adult male |
| fragment 9 | | lung cDNA |
| Target | Mm.1104 | Mus musculus ubiquitin- |
| fragment 10 | | activating enzyme E1, Chr X |

2. Oligonucleotide Immobilization

The oligonucleotide designed based on each of the target fragments was immobilized on a DNA chip. First, a commercial slide glass (Gold Seal Brand) was immersed in an alkali solution (sodium hydroxide: 50 g, purified water: 150 ml, 95% ethanol: 200 ml) at room temperature for 2 hours. Then, the slide glass was transferred into purified water, and the alkali solution was thoroughly removed by three times of rinsing with purified water. The rinsed slide glass was then immersed in a 10% aqueous solution of poly-L-lysine (Sigma) for 1 hour and then centrifuged on a centrifuge for microtiter plates at 500 rpm for 1 minute for removing the aqueous solution of poly-L-lysine. Then, the slide glass was placed in a suction thermostat and dried at 40° C. for 5 minutes, for amino group introduction onto the slide glass. Further, the amino group-carrying slide glass was immersed in 1 ml of a 1 mM dimethyl sulfoxide solution of GMBS (PIERCE) for 2 hours and then washed with dimethyl sulfoxide, whereby a maleimide group was introduced onto the slide glass surface. Then, thiol group-containing oligonucleotides were synthesized based on the sequences designed in Example 6-1, respectively. For the synthesis, an automated DNA synthesizer (Applied Biosystem model 394 DNA synthesizer) was used, and each oligonucleotide was purified by high performance liquid chromatography. A spotting solution was prepared by mixing up 1.0 μl of 2 μM oligonucleotide, 4.0 μl of HEPES buffer (N-2-hydroxyethlpiperazine-N,-2-ethanesulfonic acid; 10 mM, pH 6.5) and 5.0 μl of an additive (ethylene glycol). The thus-prepared spotting solution was spotted onto the slide glass slide using a spotter (Hitachi Software, SPBIO 2000), and the slide glass was then allowed to stand at room temperature for 2 hours to effect immobilization of the oligonucleotide on the slide glass.

3. Hybridization Method

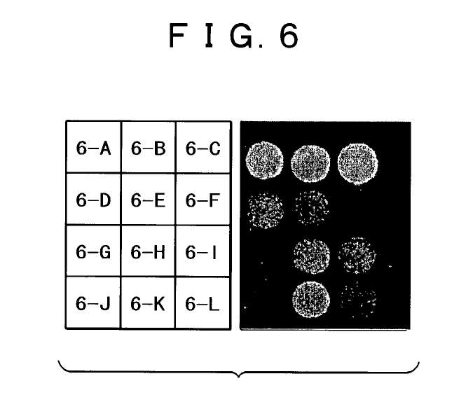

Cy5-labeled cDNA was synthesized based on the mRNA sample to serve as a control by the reverse transcription reaction using Cy5-dCTP. On the other hand, Cy3-labeled cDNA was synthesized based on the mRNA from the specifically treated sample by the reverse transcription reaction using Cy3-dCTP. Equal amounts of the Cy5-labeled cDNA and Cy3-labeled cDNA were mixed and applied to a chip with the above oligonucleotides immobilized thereon, and hybridization was carried out at 62° C. for 12 hours. After washing, visualization was effected using a scanner (GSI-Lumonics, ScanArray 5000). The results are shown in FIG. 6 as the results of an expression analysis using the DNA chip. 6-A and 6-B are expression patterns as revealed using the control DNA as a probe. 6-C, 6-D, 6-E, 6-F, 6-G, 6-H, 6-I, 6-J, 6-K and 6-J are expression patterns as found using the nucleotide sequences of target fragments 1 to 10, respectively, as probes. Differences were detected in expression levels among the respective genes, and expression patterns corresponding to the band densities respectively indicative of the expressions in FDD were obtained. Thus, the results of gene expression analysis by the FDD method agreed with those of gene expression analysis using the chip. It was thus revealed that the target fragments obtained by the FDD method could be accurately detected by the present invention. Thus, it has been verified that appropriate probes designed based on the nucleotide sequences obtained in accordance with the present invention can be applied to chip analysis and the like, further enabling rapid and accurate expression analysis of unknown and other genes.

The present invention also preferably comprises the further methods described immediately below, including:

An information providing method which comprises the steps of:

preparing a DNA fragment having a specific sequence and a first size;

cleaving the DNA fragment having the above first size at a specific sequence for obtaining information about the size of a DNA fragment having a second size as resulting from the cleavage,

predicting a gene in a gene database using the above-mentioned specific sequence and the above second size information to extract a target gene, and

cloning the above target gene and providing the cloning product.

A method of producing chips which comprises the steps of:

preparing a DNA fragment having a specific sequence and a first size,

cleaving the DNA fragment having the above first size at a specific sequence for obtaining information about the size of a DNA fragment having a second size as resulting from the cleavage,

searching for a gene in a gene database using the above-mentioned specific sequence and the above second size information to extract a target gene, and

immobilizing the above target gene on a chip.

A method of hybridization which comprises the steps of:

preparing a DNA fragment having a specific sequence and a first size;

cleaving the DNA fragment having the above first size at a specific sequence for obtaining information about the size of a DNA fragment having a second size as resulting from the cleavage,

searching for a gene in a gene database using the above-mentioned specific sequence and the above second size information to extract a target gene,

immobilizing the above target gene on a chip, and

adding, to the above chip, a solution containing a nucleotide sequence having a sequence complementary to the above target gene to thereby effect hybridization.

The present invention makes it possible to predict a gene without the steps of cloning and sequencing the relevant DNA contained in the target fragment. Thus, the method of gene analysis is simplified, and the efficiency of gene analysis is markedly improved.

Further, it becomes possible to know the names of genes for large amounts of DNA fragments in a short period of time, hence a high level of throughput can be realized, and the possibility of a novel gene being found is remarkably increased.

The present invention also facilitates the prediction and identification of target gene fragments, and can provide definitely and easily a list of search results and products of cloning of target fragments.

The foregoing invention has been described in terms of preferred embodiments. However, those skilled in the art will recognize that many variations of such embodiments exist. Such variations are intended to be within the scope of the present invention and the appended claims.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered by way of example only to facilitate comprehension of the invention and should not be construed to limit the scope thereof.

What is claimed is:

1. A method of gene prediction comprising:
preparing a DNA fragment having a first specific sequence and a first size;
cleaving the DNA fragment having said first size at said first specific sequence for obtaining information about the size of a DNA fragment having a second size resulting from the cleavage; and
searching for a gene in a gene database using said first specific sequence and said second size information to extract a target gene.

2. The method of gene prediction according to claim 1 wherein the DNA fragment having said first size is labeled at one end, and said second size information is obtained by subtracting the size between said one end and said first specific sequence from said first size.

3. The method of gene prediction according to claim 1 wherein the cleavage is carried out using a restriction enzyme or enzymes.

4. The method of gene prediction according to claim 1 wherein said first size is prepared by a differential display method.

5. The method of gene prediction according to claim 1 wherein said DNA fragment has the sequence of a gene showing a change in expression level between a patient-derived sample and a normal subject-derived sample.

6. The method of gene prediction according to claim 5 wherein said expression level results from a change with the lapse of time.

7. The method of gene prediction according to claim 1 wherein said second size information is obtained by electrophoresis.

8. The method of gene prediction according to claim 1 wherein:
a plurality of DNA fragments each having said first size and a second specific sequence are prepared;
each DNA fragment having said first size is cleaved at said second specific sequence for obtaining information about the size of a DNA fragment having a third size resulting from the cleavage; and
a search is made for a gene in said gene database using said second specific sequence and said third size information.

9. The method of gene prediction according to claim 1 wherein:
a plurality of DNA fragments each having said first size and two or more different specific sequences are prepared;
each of the plurality of DNA fragments is cleaved at said two or more different specific sequences to give a plurality of cleavage fragments;
a logical formula is formulated using the size information about said plurality of fragments as well as said two or more different specific sequences as elements thereof; and
a search is made for a gene in said gene database using said logical formula.

10. The method of gene prediction according to claim 9 wherein the logical formula contains NOT.

11. A method of gene prediction comprising the steps of:
preparing information about a first DNA fragment having a first size with a specific sequence and a known nucleotide sequence at one end thereof and information about the size of a second DNA fragment having a second size as obtained by cleavage of said first DNA fragment at said specific sequence;
conducting a homology search with respect to said known nucleotide sequence using a gene database; and
conducting a gene prediction based on said second size and said specific sequence using said gene database.

12. The method of gene prediction according to claim 1 further comprising the step of constructing a primary database in which the results of said homology search are stored, wherein said primary database is used in said prediction step.

13. The method of gene prediction according to claim 12 wherein said homology search is a step in which searching is conducted with a given reference value of homology.

14. A method of providing a list comprising the steps of:
preparing a DNA fragment having a first size and a specific sequence;
cleaving the DNA fragment having said first size at said specific sequence;
conducting a search in a gene database based on the size of the cleaved DNA fragment and on said specific sequence for extracting genes;
preparing a list of the genes extracted; and
providing said list.

15. The method of providing a list according to claim 14 wherein:
each one of a plurality of DNA fragments has a plurality of specific sequences;
each of the plurality of said DNA fragments is cleaved at each of said plurality of specific sequences to obtain a plurality of cleaved DNA fragments; and
said prediction step includes a step of searching for a gene in said gene database based on the size of each of said plurality of cleaved DNA fragments and on said plurality of specific sequences.

16. The method of providing a list according to claim 14 wherein said list of genes extracted includes a novel gene or a useful gene.

17. The method of providing a list according to claim 14 wherein:
said DNA fragment has a known nucleotide sequence; and
further comprising a step of conducting a search for said known nucleotide sequence in said gene database prior to said extraction step.

18. The method of providing a list according to claim 14 wherein said list contains the name of a gene.

19. The method of providing a list according to claim 14 wherein said list includes a nucleotide sequence.

20. The method of providing a list according to claim 14 wherein said list of genes extracted includes a novel gene or a useful gene.


SEQUENCE LISTING (10 sequences)

SEQ ID NO: 1 - DNA, 10 bp, Artificial sequence
  Known nucleotide sequence primer by OPERON TECHNOLOGY, Inc.
ggacgacaag

SEQ ID NO: 2 - DNA, 10 bp, Artificial sequence
  Known nucleotide sequence primer by OPERON TECHNOLOGY, Inc.
gtagccgtct

SEQ ID NO: 3 - DNA, 17 bp, Artificial sequence
  Oligo-d(T) primer by Nippon Flour Mills
gttttttttttttttta

SEQ ID NO: 4 - DNA, 1790 bp, Mouse
agccgctgctgctgtcgcgcagtccgctcctccgctgcagagtcgtgccctgagctcggccgacaaggctgccttcgcagccgggatcctgccagccgcgaccccagccttcgccgtcgccgcctagggcgccccaggccgcaccatggtgaaggtgacgttcaactcggcgctggcccagaaggaggccaagaaggacgagcccaagagcagcgaggaggcgctcatcgtccctccggatgccgtggcggtggattgcaaggacccgggtgacgtggttccggttggacagaggagagcgtggtgttggtgcatgtgtttcggactggccttcatgcttgctggcgtcatcctcggaggggcgtacctgtacaagtattttgctcttcagccagatgatgtgtactactgtggactaaagtacatcaaagatgacgtcatcctgaacgagccttctgcggatgccccagctgctcgctaccagacaattgaagagaacattaagatctttgaggaagacgcagtggaattcatcagtgtgcctgtaccagagtttgcggacagcgatcctgccaacattgtgcacgacttcaacaagaaactcactgcttatttggaccttaacctggacaagtgctacgtgattcctctgaacacttccatcgttatgccgcccaaaaacctgctggagctccttattaacattaaggccgggacctacctgcctcagtcctaccttatccatgagcacatggtgatcaccgaccgcatcgagaacgtggacaacctgggcttcttcatctaccgactgtgtcacgacaaggagacctacaaactgcagcgccgggaaacaattagaggtattcagaagcgggaagccagtaactgtttcaccattcggcattttgagaacaaatttgctgtggagactttaatttgttcttgagaagtcaagaaaaaacgtggggaggaattcaatgccacagcataccctgcccctctgtattttgtgcagtgattgttttttaaaatcttcttttcatgtaagtagcaaacagggcttcactgtctcctcatctcaataactcaattaaaaaccattatcttaaaaaaagaaaacaaaacctttcttttttctaagtgtggcgtctttgatgtttgaattagcaaatgtgcaggttcctagataagattcgcttctccttagagcttacctactaggaagaatctaaattgcttggaaatcactaatctggatttttgtgttaattctgcacttccatgagggaaagatgcctaaagaatagtcattcgcatatgttaaagggaccaccgtgacttgcttgtagacgctagccctgctacctagtctgttagcatttgaagtcaccgtctctactactttaattgaaatgtgccctatcttcaatgttgctttaactactttagagggtttcagccctgatgttttaatatcctaggcctctgctgtaataagattttagacaaatgtttggaatttaagaagcaactcatgttactaatttgtataggcccatatctgtggaatggaatataaatatcacaaagccatgtgatgagactgtgcgttgtttttcccataggataaaaccaaagaagtaatttggttctccatactttaaggtaatccacatacataaaaaatgaaactattttataaagtctagttctctacatgcagttataaaaatcagcttttttaaaaaataaaataagccattaattactaaaaaaaaaaaaaaaaaaaa

SEQ ID NO: 5 - DNA, 795 bp, Mouse
ggacgacaaggagacctacaaactgcagcgccgggaaacaattagaggtattcagaagcgggaagccagtaactgtttcaccattcggcattttgagaacaaatttgctgtggagactttaatttgttcttgagaagtcaagaaaaaacgtggggaggaattcaatgccacagcataccctacccctttgtattttgtgcagtgattgttttttaaaatcttcttttcatgtaagtagcaaacagggcttcactgtctcttcatctcaataactcaattaaaaaccattatcttaaaaaaagaaaacaaaacctttcttttttctaagtgtggtgtctttgatgtttgaattagcaaatgtgcaggttcctagataagattcgcttctccttagagcttacctactaggaagaatctaaattgcttggaaatcactaatctggatttttgtgttaattctgcacttccatgagggaaagatgcctaaagaatagtcattcgcatatgttaaagggaccacagtgacttgcttgtagatgctagccctgctacctagtctgttagcatttgaagtcaccttctcatactactttaattaaaatgtgccgtatcttcaatgttgctttaactactttagaggatttcagccttgatgttttaatatcctaggcctctgctgtaataagattttagacaaatgtttggaatttaagaagcaactcatgttactaatttgtatagcccatatctgtggaatggaatataaatatcacaaagcctaaaaaaaaaaaaaaa

SEQ ID NO: 6 - DNA, 431 bp, Mouse
gtagccgtctctccagctcctaacactaagtttccctatttattcacgtgtgtgtatgtatgttcatatttgtgtgtgactgtctttttcactgaacctagagcttagcaaatggctatactggctggccagcaagcacccagggtccttctgtttgcctccccggctgggattacagactcatgttgccgtgcgctggattttctatgagtgcctgggctccaaactcaggtcctaatgtttgcattgtaagcactttaacaaacgagccatctccccagtccccttagtaatctgtttagtaatctgttagaaatctgttttggttagagttatgcccatagcattggattcccttggctgtatggagggatctggaattcttggaaacaggagataaaattagaagtgaaggtaaaaaaaaaaaaaaa

SEQ ID NO: 7 - DNA, 1790 bp, Mouse; identical to SEQ ID NO: 4

SEQ ID NO: 8 - DNA, 1621 bp, Mouse
gggaaaccgcgctgcactgagccgctgctgctgtcgcgcagtccgctcctccgctgcagagtcgtgccctgagctcggccgacaaggctgccttcgcagccggggatcctgccagccgcgaccccagccttcgccgtcgccgcctagggcgccccaggccgcaccatggtgaaggtgacgttcaactcggcgctggcccagaaggaggccaagaaggacgagcccaagagcagcgaggaggcgctcatcgtccctccggatgccgtggcggtggattgcaaggacccgggtgacgtggttccggttggacagaggagagcgtggtgttggtgcatgtgtttcggactggccttcatgcttgctggcgtcatcctcggaggggcgtacctgtacaagtattttgctcttcagccagatgatgtgtactactgtggactaaagtacatcaaagatgacgtcatcctgaacgagccttctgcggatgccccagctgctcgctaccagacaattgaagagaacattaagatctttgaggaagacgcagtggaattcatcagtgtgcctgtaccagagtttgcggacagcgatcctgccaacattgtgcacgacttcaacaagaaactcactgcttatttggaccttaacctggacaagtgctacgtgattcctctgaacacttccatcgttatgccgcccaaaaacctgctggagctccttattaacattaaggccgggacctacctgcctcagtcctaccttatccatgagcacatggtgatcaccgaccgcatcgagaacgtggacaacctgggcttcttcatctaccgactgtgtcacgacaaggagacctacaaactgcagcgccgggaaacaattagaggtattcagaagcgggaagccagtaactgtttcaccattcggcattttgagaacaaatttgctgtggagactttaatttgttcttgagaagtcaagaaaaaacgtggggaggaattcaatgccacagcataccctgcccctttgtattttgtgcagtgattgttttttaaaatcttcttttcatgtaagtagcaaacagggcttcactgtctcttcatctcaataactcaattaaaaaccattatcttaaaaaaagaaaacaaaacctttcttttttctaagtgtggtgtctttgatgtttgaattagcaaatgtgcaggttcctagataagattcgcttctccttagagcttacctactaggaagaatctaaattgcttggaaatcactaatctggatttttgtgttaattctgcacttccatgagggaaagatgcctaaagaatagtcattcgcatatgttaaagggaccacagtgacttgcttgtagatgctagccctgctacctagtctgttagcatttgaagtcaccttctcatactactttaattaaaatgtgccgtatcttcaatgttgctttaactactttagaggatttcagccttgatgttttaatatcctaggcctctgctgtaataagattttagacaaatgtttggaatttaagaagcaactcatgttactaatttgtatagcccatatctgtggaatggaatataaatatcacaaagccaaaaaaaaa

SEQ ID NO: 9 - DNA, 1577 bp, Mouse
ctccgctgcagagtcgtgccctgagctcggccgacaaggctgccttcgcagccgggatcctgccagccgcgaccccagccttcgccgtcgccgcctagggcgccccaggccgcaccatggtgaaggtgacgttcaactcggcgctggcccagaaggaggccaagaaggacgagcccaagagcagcgaggaggcgctcatcgtccctccggatgccgtggcggtggattgcaaggacccgggtgacgtggttccggttggacagaggagagcgtggtgttggtgcatgtgtttcggactggccttcatgcttgctggcgtcatcctcggaggggcgtacctgtacaagtattttgctcttcagccagatgatgtgtactattgtggactaaagtacatcaaagatgacgtcatcctgaacgagccttctgcggatgccccagctgctcgctaccagacaattgaagagaacattaagatctttgaggaagacgcagtggaattcatcagtgtgcctgtaccagagtttgcggacagcgatcctgccaacattgtgcatgacttcaataagaaactcactgcttatttggaccttaacctggacaagtgctacgtgattcctctgaacacttccatcgttatgccgcccaaaaacctgctggagctccttattaacattaaggccgggacctacctgcctcagtcctaccttatccatgagcacatggtgatcaccgaccgcatcgagaacgtggacaacctgggcttcttcatctaccgactgtgtcacgacaaggagacctacaaactgcagcgccgggaaacaattagaggtattcagaagcgggaagccagtaactgtttcaccattcggcattttgagaacaaatttgctgtggagactttaatttgttcttgagaagtcaagaaaaaacgtggggaggaattcaatgccacagcataccctgcccctttgtattttgtgcagtgattgttttttaaaatcttcttttcatgtaagtagcaaacagggcttcactgtctcttcatctcaataactcaattaaaaaccattatcttaaaaaaagaaaacaaaacctttcttttttctaagtgtggtgtctttgatgtttgaattagcaaatgtgcaggttcctagataagattcgcttctccttagagcttacctactaggaagaatctaaattgcttggaaatcactaatctggatttttgtgttaattctgcacttccatgagggaaagatgcctaaagaatagtcattcgcatatgttaaagggaccaccgtgacttgcttgtagacgctagccctgctacctagtctgttagcatttgaagtcaccgtctctactactttaattgaaatgtgccctatcttcaatgttgctttaactactttagagggtttcagccctgatgttttaatatcctaggcctctgctgtaataagattttagacaaatgtttggaatttaagaagcaactcatgttactaatttgtatagcccatatctgtggaatggaatataaatatcacaaagccaaaaaaaaaaaaaaa

SEQ ID NO: 10 - DNA, 1583 bp, Mouse
ggctgtcgcgcagtccgctcctccgctgcagagtcgtgccctgagctcggccgacaaggctgccttcgcagccgggatcctgccagccgcgaccccagccttcgccgtcgccgcctagggcgccccaggccgcaccatggtgaaggtgacgttcaactcggcgctggcccagaaggaggccaagaaggacgagcccaagagcagcgaggaggcgctcatcgtccctccggatgccgtggcggtggattgcaaggacccgggtgacgtggttccggttggacagaggagagcgtggtgttggtgcatgtgtttcggactggccttcatgcttgctggcgtcatcctcggaggggcgtacctgtacaagtattttgctcttcagccagatgatgtgtactactgtggactaaagtacatcaaagatgacgtcatcctgaacgagccttctgcggatgccccagctgctcgctaccagacaattgaagagaacattaagatctttgaggaagacgcagtggaattcatcagtgtgcctgtaccagagtttgcggacagcgatcctgccaacattgtgcacgacttcaacaagaaactcactgcttatttggaccttaacctggacaagtgctacgtgattcctctgaacacttccatcgttatgccgcccaaaaacctgctggagctccttattaacattaaggccgggacctacctgcctcagtcctaccttatccatgagcacatggtgatcaccgaccgcatcgagaacgtggacaacctgggcttcttcatctaccgactgtgtcacgacaaggagacctacaaactgcagcgccgggaaacaattagaggtattcagaagcgggaagccagtaactgtttcaccattcggcattttgagaacaaatttgctgtggagactttaatttgttcttgagaagtcaagaaaaaacgtggggaggaattcaatgccacagcataccctgcccctttgtattttgtgcagtgattgttttttaaaatcttcttttcatgtaagtagcaaacagggcttcactgtctcttcatctcaataactcaattaaaaaccattatcttaaaaaaagaaaacaaaacctttcttttttctaagtgtggtgtctttgatgtttgaattagcaaatgtgcaggttcctagataagattcgcttctccttagagcttacctactaggaagaatctaaattgcttggaaatcactaatctggatttttgtgttaattctgcacttccatgagggaaagatgcctaaagaatagtcattcgcatatgttaaagggaccacagtgacttgcttgtagatgctagccctgctacctagtctgttagcatttgaagtcaccttctcatactactttaattaaaatgtgccgtatcttcaatgttgctttaactactttagaggatttcagccttgatgttttaatatcctaggcctctgctgtaataagattttagacaaatgtttggaatttaagaagcaactcatgttactaatttgtatagcccatatctgtggaatggaatataaatatcacaaagcc